(19) United States
(12) Patent Application Publication
(10) Pub. No.: US 2008/0298614 A1
(43) Pub. Date: Dec. 4, 2008

(54) System for and Method of Offering an Optimized Sound Service to Individuals within a Place of Business

(75) Inventors: John Cronin, Jericho, VT (US); Mark Burrows, Princeton, NJ (US); Tom Hunt, Shelburne, VT (US); Nancy Edwards, Jericho, VT (US); Justin Kunz, South Burlington, VT (US)

Correspondence Address: MCCARTER & ENGLISH, LLP, FOUR GATEWAY CENTER, 100 MULBERRY STREET, NEWARK, NJ, 07102, US

(73) Assignee: Johnson & Johnson Consumer Companies, Inc., Skillman, NJ (US)

(21) Appl. No.: 11/570,489

(22) PCT Filed: Jun. 9, 2005
(86) PCT No.: PCT/US05/20266

§ 371 Date: Aug. 4, 2008

Related U.S. Application Data
(60) Provisional application No. 60/579,367, filed on Jun. 14, 2004.

(51) Int. Cl. H04R 25/00; A61B 5/00; H04R 29/00

(52) U.S. Cl. 381/314; 381/315; 381/312

(57) ABSTRACT

The present invention relates to optimizing sound for a person based on his or her location and hearing profile. More particularly, the present invention relates to personalizing the sound of an event or performance by examining the technical specifications of an individual's hearing aid (which is provided by a third party) and the acoustics of an establishment. Using this information, a place of business alters the settings of each hearing aid device to optimize the sound for each individual.

### CROSS-REFERENCED TO RELATED APPLICATION

This application claims the benefit of U.S. Provisional Application No. 60/579,367 filed Jun. 14, 2004, assigned to the assignee of this application and incorporated by reference herein.

### FIELD OF THE INVENTION

The present invention relates to optimizing sound for a person based on his or her location and hearing profile. More particularly, the present invention relates to personalizing the sound of an event or performance by examining the technical specifications of an individual's hearing aid (which is provided by a third party) and the acoustics of the venue. Using this information, a place of business alters the settings of each hearing aid device to optimize the sound for each individual.

### BACKGROUND OF THE INVENTION

More than 25 million Americans have hearing loss, including one of four people older than 65. Hearing loss may come from infections, strokes, head injuries, some medicines, tumors, other medical problems, or even excessive earwax. It can also result from repeated exposure to very loud noise, such as music, power tools, or jet engines. Changes in the way the ear works as a person ages can also affect hearing.

For most people who have a hearing loss, there are ways to fix the problem. If an individual has trouble hearing, that individual can visit a doctor or hearing health-care professional to find out if he or she has a hearing loss and if so, determine a remedy. The U.S. Food and Drug Administration (FDA), like governing bodies in other countries, has rules to make sure that treatments for hearing loss—medicines, hearing aids, and other medical devices—are tried and tested.

If a hearing test shows that the individual has a hearing loss, there may be one or more ways to treat it. Possible treatments include medication, surgery, or a hearing aid. Hearing aids can usually help hearing loss that involves damage to the inner ear. This type of hearing loss is common in older people as part of the aging process. However, younger people can also develop hearing loss from infections or repeated exposure to loud noises.

In a well-known method of testing hearing loss in individuals, the threshold of the individual's hearing is typically measured using a calibrated sound-stimulus-producing device and calibrated headphones. The measurement of the threshold of hearing takes place in an isolated sound room where there is very little audible ambient noise. The sound-stimulus-producing device and calibrated headphones used in the testing are known in the art as an audiometer.

A professional audiologist performs a professional test by using the audiometer to generate pure tones at various frequencies between 125 Hz and 12,000 Hz that are representative of a variety of frequency bands. These tones are transmitted through the headphones of the audiometer to the individual being tested. The intensity or volume of the pure tones is varied until the individual can barely detect the presence of the tone. For each pure tone, the intensity at which the individual can barely detect the presence of the tone is known as the individual's air conduction threshold of hearing.

Once the hearing test determines how to compensate for the individual's hearing loss, compensation factors are sent to a hearing aid manufacturer to program the digital signal processor (DSP) of a hearing aid. The hearing aid is manufactured and programmed before being sent to the audiologist. The audiologist then schedules an appointment with the individual, in which the audiologist physically fits the hearing aid and makes electrical adjustments if needed. These adjustments often include helping the user set the volume control and any other adjustments the hearing aid allows. The adjustments to the hearing aid are made based on the results of another hearing test conducted by the audiologist upon the user with the hearing aid in his or her ear. The repeated hearing aid test may in fact require further frequency versus amplitude adjustments that are not possible after the manufacturer has determined the settings. These adjustments are often necessary because the acoustical differences between a hearing test conducted with headphones and the same hearing test conducted with a programmed hearing aid cause the individual's responses to vary.

This is overcome in U.S. Pat. No. 6,319,020, incorporated by reference herein, which describes a device for coupling a programming connector to a programmable hearing aid comprising an electrode coupled to a corresponding conductor of the programming connector, wherein the electrode is biased to maintain contact with a conductive surface in the hearing aid. The coupling device is adapted to engage within a receiver module of a CIC hearing device. Data from an outside source, such as a computer, can thereby be easily transferred through the programming connector to circuitry within the hearing device.

As demonstrated in the prior art, highly sophisticated programmable hearing aid fitting systems have been developed to accurately and satisfactorily fit a hearing aid on a user. However, the prior art fitting systems are largely complicated and time consuming. Therefore, it is assumed that a particular user may undergo such a fitting process only once. Very little has been done in the prior art to continuously monitor and calibrate an already fitted hearing aid according to the user's surrounding environment. For example, two different users can be diagnosed with exactly the same hearing loss and can be fitted with hearing aids that are programmed very similarly. The first user works as a referee for a professional football league and the second user works in a nursing home for retired football players. These two individuals are exposed to the same words at highly differing sound frequencies and amplitudes at a given time. When testing and fitting the hearing aids, both users responded positively to being able to hear the word “touchdown” at normally spoken frequencies. However, after the hearing aids were fitted to the users, the first user experienced difficulties with the same word “touchdown” spoken in a football stadium at higher frequencies and amplitudes. Therefore, what is needed is a way of recalibrating the first user's hearing aid after its initial fitting and after the individual has had time to test the hearing aid with respect to his or her environment.

Moreover, the first user could experience difficulties in multiple environments, such as his or her daytime job in a noisy football stadium and a nighttime job in a quiet environment as a security guard. Therefore, what is needed is a way to easily and repeatedly calibrate the hearing aid according to the user's specific hearing needs, such as multiple environments of use.

Providing a way to easily and repeatedly calibrate a hearing aid according to the multiple environments of a user may prove to be a good business opportunity. For example, a football stadium can provide special “plug-in” seats for hearing aid users. These “plug-in” seats have outlets for hearing aid users to calibrate their hearing aids for the duration of a football game. In turn, the football stadium owner can charge a premium price for the special “plug-in” seats. What is needed is a process to convert a way to repeatedly calibrate a hearing aid according to the user's specific needs into a business transaction.

It is therefore it is an object of the present invention to demonstrate a way to recalibrate a hearing aid after the user is initially fitted with the hearing aid and the user has had some time to test out the hearing aid with respect to his or her environment.

It is another object of the present invention to demonstrate a way to easily and repeatedly calibrate a hearing aid according to the user's specific hearing needs, such as multiple environments of use.

It is yet another object of the present invention to illustrate a process to convert a way to repeatedly calibrate a hearing aid according to the user's specific needs into a business transaction.

### SUMMARY OF THE INVENTION

The present invention is a system for and method of offering an optimized sound service to an individual within a place of business. This invention includes a method of providing hearing information about individuals to any number of establishments. Coupled with hearing data collected about the individual's physical location, such as the location of the individual's seat, this invention also provides an establishment with a system for optimizing the digital signal processor (DSP) of an individual's hearing aid. Finally, this invention provides a method of generating revenue by offering such a system.

Thus, the present invention provides for a method of adjusting hearing aid operation based on acoustic characteristics of a location comprising the steps:
(a) providing acoustic characterization data corresponding to a location;
(b) providing a database of hearing loss profiles associated with a respective plurality of individuals using hearing aids, wherein the hearing loss profiles include respective digital signal processor (“DSP”) correction factors for use by a DSP of a hearing aid; and
(c) computing adjusted DSP correction factors for a user based on the acoustic characterization data corresponding to a location.

In a preferred embodiment, the method further comprises the step of:
(d) transmitting the adjusted DSP correction factors as a DSP programming signal for programming a DSP of a hearing aid, wherein the DSP of the hearing aid modifies input audio signals using the adjusted DSP correction factors.

In a further preferred embodiment of the method, the location includes a plurality of regions and the acoustic characterization data includes region characterization data for the respective regions.

In a further preferred embodiment of the method, at least one of the acoustic characterization data and the hearing loss profile database is accessible over a communications network.

In a further preferred embodiment, the method includes requiring payment of a fee before performing at least one of steps (a), (b) and (c).

In a further preferred embodiment, the method includes identifying a favored acoustic region (e.g., best seat) in the location based on the acoustic characterization data and the hearing loss profile of an individual using a hearing aid.

In a still further embodiment, the method includes providing access to acoustic characterization data and the hearing loss profile database over a communications network. The location comprises a plurality of regions having distinct acoustic properties and the acoustic characterization data comprises regional data corresponding thereto. The location further comprises a connection interface (such as an input/out device connection or a wireless input/output connection) at each of said regions (or identifiable as emanating from a particular region) for allowing a data connection between the hearing aid and the communications network, and further comprising connecting the hearing aid to the communications network when the hearing aid is located within a particular region within the location, computing the adjusted DSP correction factor based on characterization data associated with that region and transmitting the adjusted DSP correction factor as a DSP programming signal for programming the DSP of the hearing aid, wherein the DSP of the hearing aid modifies input audio signals using the adjusted DSP correction factors.

### BRIEF DESCRIPTION OF THE DRAWINGS

FIG. 1 illustrates a group of establishments using a computer and the Internet to connect to a centralized database containing hearing data about individuals.

FIG. 2 illustrates a basic system for receiving sound, processing it through a mixing board and computer, and using an amplifier to play the sound through a speaker.

FIG. 3 illustrates a basic computer system containing a DSP, a program, and local storage.

FIG. 4 illustrates a floor plan of an establishment, showing a variety of hearing types within different sections of the establishment.

FIG. 5 illustrates a flow diagram of a process used by an establishment for optimizing the sound for an individual.

FIG. 6 illustrates a flow diagram of a business transaction taking place between a customer, a place of business, and a third-party hearing services provider.

### DESCRIPTION OF THE INVENTION

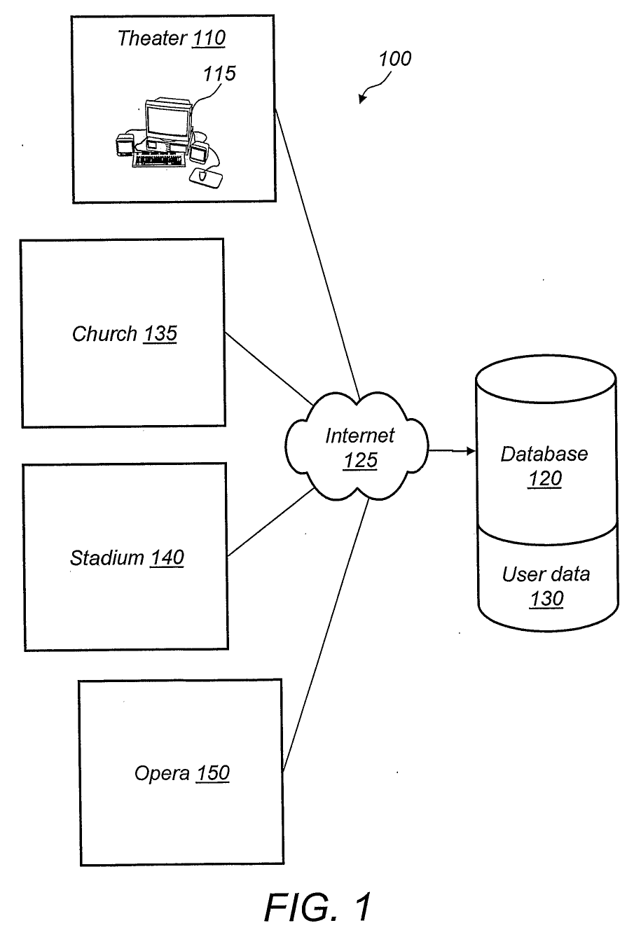

FIG. 1 is a diagram of a system 100 for providing establishments with individuals' hearing information consisting of a theater 110, a conventional computer 115, a database 120, the Internet 125, an example of user data 130, a church 135, a stadium 140, and an opera 150.

Theater 110 is an example of an establishment having signed up as a client of hearing health system 100. The establishment is interested in hearing profile information of individuals so as to optimize sound for those individuals. Computer 115 resides within each establishment. Computer 115 accesses database 120 using Internet 125. Database 120 belongs to a third party and resides in a remote location separate from theater 110. Database 120 contains hearing information about individuals in the form of user data 130. For example, user data 130 includes specific technical information about an individual's hearing aid, such as specific frequencies and amplitudes that the person has trouble hearing. Computer 115 allows a user or program residing at theater 110 to access user data 130 with appropriate security and user permissions.

Several other examples of establishments are shown. Church 135, stadium 140, and opera 150 also have access to the same hearing health database using their own computers 115 and Internet 125.

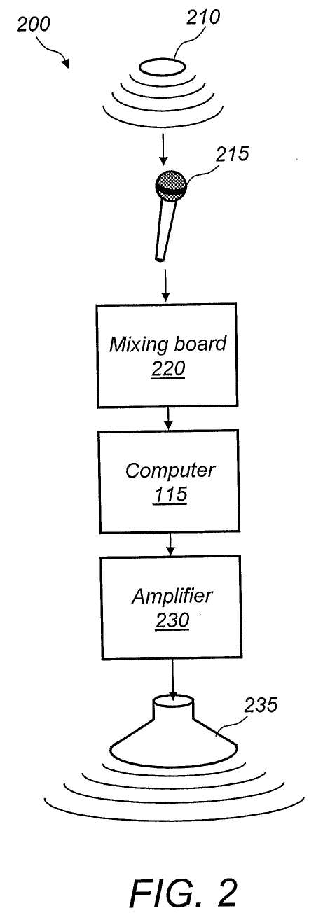

FIG. 2 is a diagram of a system 200 for amplifying sound consisting of a sound source 210, a microphone 215, a mixing board 220, computer 115, an amplifier 230, and a speaker 235.

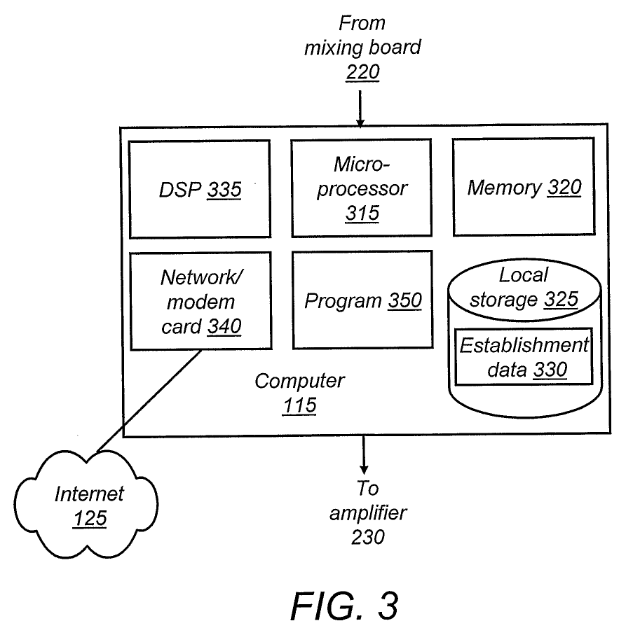

System 200 shows a common setup used to amplify sound to a large group or audience. In this case, system 200 exists within theater 110 as described in FIG. 1. Sound source 210 is the voice of an actor or actress performing on stage. This person has some method of inputting his or her voice into system 200, commonly microphone 215 attached to his or her body or near the stage. Microphone 215 is typically input to mixing board 220. Mixing board 220 performs a number of functions, including the amplification and combining of sounds. Mixing board 220 connects to computer 115. Computer 115 takes the sound from mixing board 220 and passes it to amplifier 230. Using a program, computer 115 performs a number of different functions with the sound, including many of the functions for which mixing board 220 is responsible. Computer 115 is described in more detail in FIG. 3. Amplifier 230 increases the decibel level, or loudness, of the original sound source 210 and passes the sound to speaker 235, which transmits the amplified sound to an audience.

FIG. 3 is a block diagram of computer 115, including a microprocessor 315, a memory 320, a local storage 325 containing a quantity of establishment data 330, a DSP 335, a network/modem card 340, Internet 125 and program 350.

Computer 115 is necessary for the hearing information retrieval process explained in reference to FIG. 1 and the sound amplification process explained in reference to FIG. 2. Microprocessor 315 of computer 115 processes each operation of the system. Information is temporary held in memory 320 before being output, permanently stored, or redistributed to other parts of computer 115. Local storage 325 is one example of this permanent storage. For example, information about an individual is accessed via Internet 125 as explained in reference to FIG. 1; and the information is then written to a hard disk drive contained within computer 115. To access this data at a later time, a user can simply retrieve the information from the hard disk.

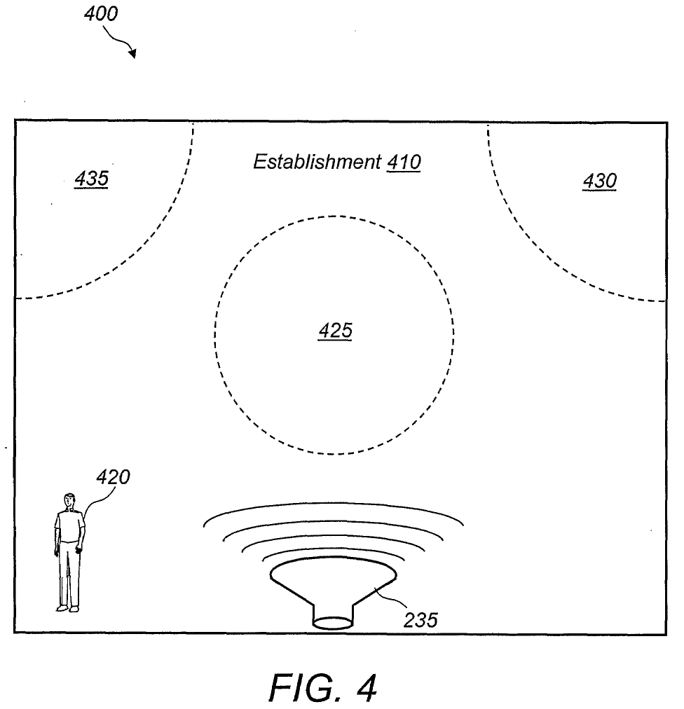

Computer 115 utilizes local storage 325 to hold establishment data 330. A process for collecting establishment data 330 is explained further with reference to FIG. 4. DSP 335 is also contained within computer 115. DSP 335 takes sound source 210 and, in conjunction with program 350, analyzes the frequency versus amplitude spectrum of the sound or voice. Program 350 processes the sound data from DSP 335 to determine the differences between normal spectrums and abnormal spectrums. For example, if sound source 210 plays low frequency sounds in a particular area of the establishment that has abnormal attenuation, this would be noted in the program. Given such information, a person with low-frequency hearing loss (even with a hearing aid) could arrange to sit in a different area, as sound in that particular location would not be optimal. Even if a person with low-frequency hearing loss were to sit in that area, the individual's hearing aid could be programmed, upon entering the establishment, with additional amplification in the low frequency range beyond the baseline amplification programmed into the hearing aid. This collection of establishment data 330, along with knowledge of individual using the hearing aid, allows for optimized seating location, automatic hearing aid programming, or both.

Network/modem card 340 allows computer 115 to connect to Internet 125. Computer 115 uses Internet 125 to remotely access information from database 120, then uses software program 350 to analyze this information. For example, program 350 would allow input of establishment data 330 and analysis of user data 130 with sound source 210 to optimize the sound for each individual.

FIG. 4 illustrates a system 400 for collecting establishment data 330, consisting of an establishment 410, speaker 235, a sound engineer 420, an area 425 that sustains all frequencies, an area 430 that sustains notch frequencies, and an area 435 that sustains low frequencies.

System 400 shows an establishment 410 such as theater 110 as described with reference to FIG. 1. Establishment 410 wants to determine the acoustics of the building to help them optimize the properties of sound. Speaker 235 sends the amplified sound to an audience and, in this example, is located at one side of establishment 410. Using a device such as an oscilloscope, sound engineer 420 performs a series of tests in different areas of establishment 410. These tests determine information such as the effect on frequencies and amplitudes of sound in certain areas of the building. In this example, area 425 is centrally located with respect to speaker 235. Sound engineer 420 may find that all frequencies of sound provide normal amplitudes of hearing for individuals in this region. Therefore, people seated in area 425 experience normal hearing. In area 430, located towards the back right of establishment 410, there may be interference with other sounds such as traffic or people in the lobby, providing low amplitudes at specific frequencies. Therefore, people seated in area 430 have trouble hearing softer sounds or during times of interference. Area 435 illustrates yet another region of establishment 410 that could experience a change in frequencies or amplitudes from the quality of the original sound.

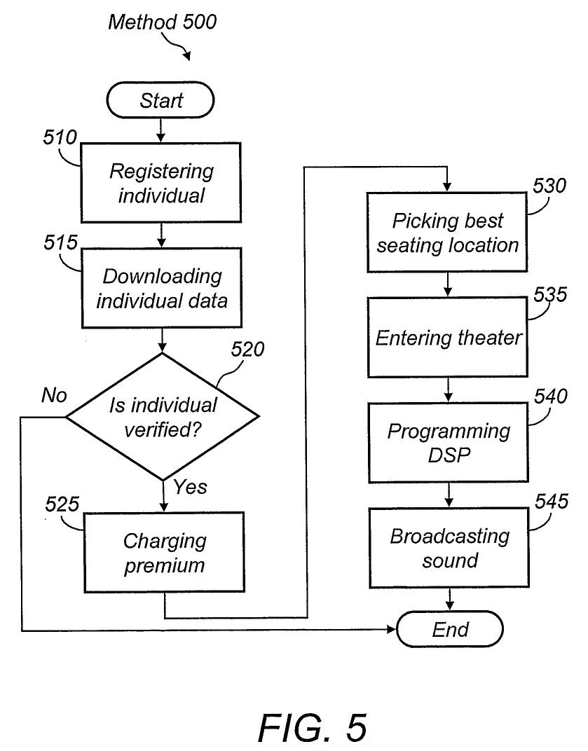

FIG. 5 illustrates a method 500 of a process used by an establishment for optimizing the sound for an individual. In this method, a person is attending a performance at an establishment and they would like to take advantage of sound optimization. Method 500 includes the steps of:

### Step 510: Registering Individual

In this step, an individual registers with establishment 410 to receive sound optimization. Ideally, this step happens at the time of ticket purchase, but could also be offered in numerous other ways before the event. If the individual buys tickets at a box office, an employee could offer this service by asking directly. Registration could be as simple as writing his or her name on a piece of paper. Preferably, the employee would input the individual using program 350 as described with reference to FIG. 3. This step is crucial to having a list of individuals, either on paper or stored electronically, who will take advantage of enhanced sound. Method 500 proceeds to step 515.

### Step 515: Downloading Individual Data

In this step, establishment 410 uses system 100 to download hearing information about individuals registered for the service. If a list of registered individuals is stored electronically, this list can be compared with the list of individuals in user data 130 on database 120. Method 500 proceeds to step 520.

### Step 520: Is Individual Verified?

In this decision step, establishment 410 must verify that the registrant in step 510 has a profile in user data 130. A piece of software residing on database 120 could display a message or list of individuals who do not have a profile stored in user data 130. For each individual located in user data 130, information is transmitted over Internet 125 to computer 115. If an individual is verified, method 500 proceeds to step 525. If not, method 500 ends.

### Step 525: Charging Premium

In this step, an individual that has signed up for sound optimization and has been verified in user data 130 is charged a premium for this service. Ideally, this happens at the time of ticket purchase, but the premium could also be collected at a box office or other location before the event.

### Step 530: Picking Best Seating Location

In this step, a specific seat can be picked for the individual because the seat would provide appropriate acoustics based on his or her personal hearing profile. Seat selection could be done either manually by an employee or automatically by a piece of software. After comparing the individual's downloaded hearing profile from step 515 and establishment 410's sound information as determined by system 400, a seat could be assigned.

### Step 535: Entering Theater

In this step, the individual enters establishment 410 and sits in the predetermined location as described in step 530.

### Step 540: Programming DSP

In this step, if program 350 has determined that it is possible to program the individual's hearing aid as well as or independent of his or her seating, the individual presents his or her hearing aid to staff of establishment 410 upon entering, and the DSP in the individual's hearing aid is independently programmed. This can be accomplished in a number of different ways, such as wirelessly (if that capability exists) programming the code to access the hearing aid and to enter the new hearing aid data for the DSP. Ether hearing aids are not able to be programmed wirelessly, so the individual must bring his or her connector to connect the hearing aid to computer 115.

### Step 545: Broadcasting Sound

In this step, establishment 410 broadcasts sound to the audience using system 200.

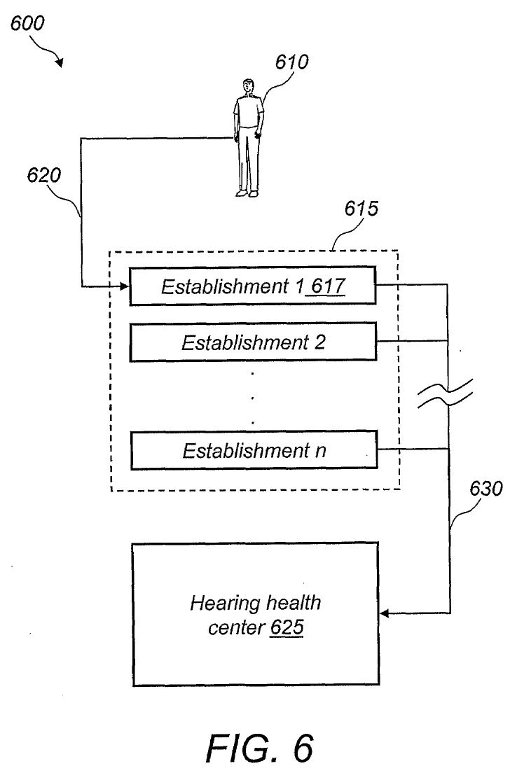

FIG. 6 illustrates a system 600 for conducting a business transaction based on the systems and method of the present invention. System 600 includes a customer 610, a group of establishments 615, a customer premium 620, an establishment 1 617, a hearing health center 625, and a client premium 630.

System 600 is the business transaction among three parties participating in the hearing optimization service. Customer 610 pays customer premium 620 in order to take advantage of this service offered by any number of group of establishments 615. In this example, customer 610 pays a nominal fee to establishment 1 617. Individual customers 610 pay a fee on an individual basis to each establishment.

Each establishment in group of establishments 615 pays client premium 630 to receive hearing information about customers 610 who desire the service. This allows the business to optimize the sound for each individual's hearing device. Client premium 630 is paid to the company that owns and maintains hearing health center 625 and database 120 storing user data 130 in individual profiles.

1. A method for optimizing sound service to an individual customized to a specific location, comprising the steps of:
providing acoustic characterization data corresponding to a location,
providing access to a database of hearing loss profiles associated with a respective plurality of individuals using hearing aids, wherein the hearing loss profiles include respective digital sound processor (“DSP”) correction factors for use by a DSP of a hearing aid; and
computing an adjusted DSP correction factor for a user based on the acoustic characterization data corresponding to the location.

2. The method of claim 1, further comprising transmitting the adjusted DSP correction factor as a DSP programming signal for programming a DSP of a hearing aid, wherein the DSP of the hearing aid modifies input audio signals using the adjusted DSP correction factors.

3. The method of claim 1, wherein the location comprises a plurality of regions having distinct acoustic properties and the acoustic characterization data comprises regional data corresponding thereto, and further comprising computing the adjusted DSP correction factor based on characterization data associated with a particular region within the location.

4. The method of claim 1, wherein at least one of the acoustic characterization data and the hearing loss profile database is accessible over a communications network.

5. The method of claim 3, further comprising determining a favored acoustic region for an individual within a location based on the regional data and the hearing loss profile of the individual.

6. The method of claim 1, wherein the acoustic characterization data and the hearing loss profile database are accessible over a communications network, and wherein the location comprises a plurality of regions having distinct acoustic properties and the acoustic characterization data comprises regional data corresponding thereto, the location further comprising a connection interface at each of said regions for allowing a data connection between the hearing aid and the communications network, and further comprising connecting the hearing aid to the communications network when the hearing aid is located within a particular region within the location, computing the adjusted DSP correction factor based on characterization data associated with that region and transmitting the adjusted DSP correction factor as a DSP programming signal for programming the DSP of the hearing aid, wherein the DSP of the hearing aid modifies input audio signals using the adjusted DSP correction factors.

7. The method of claim 1, further comprising the step of processing a user fee prior to prior to performing one or more of the steps.

8. A system for optimizing sound service to an individual customized to a specific location, comprising:
acoustic characterization data corresponding to a location,
a database of hearing loss profiles associated with a respective plurality of individuals using hearing aids, wherein the hearing loss profiles include respective digital sound processor (“DSP”) correction factors for use by a DSP of a hearing aid; and
a processing means for accessing the hearing loss profile of an individual and computing an adjusted DSP correction factor for that individual based on the acoustic characterization data corresponding to the location.

9. The system of claim 8, further comprising a means for transmitting the adjusted DSP correction factor as a DSP programming signal for programming a DSP of a hearing aid, wherein the DSP of the hearing aid modifies input audio signals using the adjusted DSP correction factors.

10. The system of claim 8, wherein the location comprises a plurality of regions having distinct acoustic properties and the acoustic characterization data comprises regional data corresponding thereto, wherein the processor comprises means for computing the adjusted DSP correction factor based on characterization data associated with a particular region within the location.

11. The system of claim 8, wherein at least one of the acoustic characterization data and the hearing loss profile database is accessible over a communications network.

12. The system of claim 10, wherein the processor further comprises means for determining a favored acoustic region for an individual within a location based on the regional data and the hearing loss profile of the individual.

13. The system of claim 8, wherein the acoustic characterization data and the hearing loss profile database are accessible over a communications network, and wherein the location comprises a plurality of regions having distinct acoustic properties and the acoustic characterization data comprises regional data corresponding thereto, the location further comprising a connection interface at each of said regions for allowing a data connection between the hearing aid and the communications network, the processor further comprising means for computing the adjusted DSP correction factor based on characterization data associated with that region and transmitting the adjusted DSP correction factor as a DSP programming signal for programming the DSP of the hearing aid, wherein the DSP of the hearing aid modifies input audio signals using the adjusted DSP correction factors.

14. The system of claim 8, further comprising means for processing a user fee prior to performing one or more of the steps.

15. Method for optimizing sound transmission to an individual, comprising the steps of:
registering an individual who uses a hearing aid with an establishment for the purpose of receiving sound optimization;
accessing stored hearing profile information data relative to the individual;
assessing a fee relative to the individual;
determining a best seating location for the individual based on the hearing profile information for the individual and audio characterization information regarding the establishment; and
programming the digital sound processor (“DSP”) of the individual's hearing aid relative to the best seating location

16. The method of claim 15, further comprising the step of, after the accessing step, verifying that the individual is registered.

17. The method of claim 15, further comprising the step of, after the determining step but prior to the programming step, the individual entering the establishment and going to the best seat location.

18. The method of claim 15, further comprising the step of the establishment providing an audio performance to the individual within the establishment.
